(19) United States
(12) Patent Application Publication
(10) Pub. No.: US 2022/0093221 A1
(43) Pub. Date: Mar. 24, 2022

(54) SYSTEM FOR ASSESSING DRUG EFFICACY AND RESPONSE OF A PATIENT TO THERAPY

(71) Applicant: Gus J. SLOTMAN, Moorestown, NJ (US)

(72) Inventor: Gus J. SLOTMAN, Moorestown, NJ (US)

(21) Appl. No.: 17/538,141

(22) Filed: Nov. 30, 2021

Related U.S. Application Data
(63) Continuation of application No. 15/619,683, filed on Jun. 12, 2017.
Application No. 15/619,683 is a continuation-in-part of application No. 14/068,595, filed on Oct. 31, 2013, now abandoned.
Application No. 14/068,595 is a continuation-in-part of application No. 13/110,265, filed on May 18, 2011, now Pat. No. 8,577,620.
Application No. 13/110,265 is a continuation-in-part of application No. 12/749,977, filed on Mar. 30, 2010, now abandoned.
Application No. 12/749,977 is a continuation of application No. 12/056,367, filed on Mar. 27, 2008, now abandoned.

(51) Int. Cl. G16H 10/20; G16H 20/10

(52) U.S. Cl. CPC G16H 10/20; G16H 50/70; G16H 20/10

(57) ABSTRACT

A system for identifying, monitoring and matching patients with appropriate treatments using a systemic mediator-associated physiologic test profile are provided. The system of the present invention increases the likelihood of demonstrating clinical efficacy in clinical trial datasets.

### INTRODUCTION

This application is a is a continuation of U.S. patent application Ser. No. 15/619,683, filed Jun. 12, 2017, which is a continuation-in-part of U.S. patent application Ser. No. 14/068,595, filed Oct. 31, 2013, now abandoned, which is a continuation-in-part of U.S. patent application Ser. No. 13/110,265, filed May 18, 2011, now U.S. Pat. No. 8,577,620, which is a continuation-in-part of U.S. patent application Ser. No. 12/749,977, filed Mar. 30, 2010, now abandoned, which is a continuation of U.S. patent application Ser. No. 12/056,367, filed Mar. 27, 2008, now abandoned, which are incorporated herein by reference in their entireties.

### BACKGROUND OF THE INVENTION

Since 1982, clinical trials of new drugs for sepsis have used, virtually unaltered, the entry criteria from the Solu-Medrol (methyprednisolone sodium succinate) study (Bone, et al. (1987) N. Engl. J. Med. 317:653-659). The Solu-Medrol definitions were first published in the report of that clinical trial's results. Subsequently, the placebo results were reported as sepsis syndrome (Bone, et al. (1989) Crit Care Med. 17:389-393). Later, they were codified into medical culture by the American College of Chest Physicians/Society of Critical Care Medicine (ACCP/SCCM) Consensus Conference on sepsis (Bone, et al. (1992) Chest 101:1644-1655). Since the ACCP/SCCM Consensus Conference, sepsis definitions were published (Bone, et al. (1992) supra), and they have been used almost exclusively as the entry criteria for sepsis clinical trials. Unfortunately, in every sepsis clinical trial that has enrolled patients under those definitions, the study drug has failed to reduce septic mortality. Even the large investigations of an anti-tumor necrosis factor (TNF) antibody (Pulmonary Reviews.com (2000) vol. 5) and recombinant activated protein C (XIGRIS; Bernard, et al. (2001) N. Engl. J. Med. 344:699-709), while statistically significant, did not reduce septic mortality to levels that changed standards of care. The anti-TNF antibody was not approved by the Food and Drug Administration (FDA), and XIGRIS has been underutilized in the medical market.

Prospective, randomized, double-blind, placebo-controlled clinical trials are accepted universally as the highest level of scientific testing for potentially therapeutic molecules in sepsis. From the accumulated sepsis clinical trial data, then, the reasonable conclusion would be that the new drugs studied simply had no beneficial effects. However, it is also possible that novel sepsis therapies have failed to reduce septic mortality because they were not tested in a study population that was responsive to their biological effects. One might speculate that clinical trial entry criteria based on the ACCP/SCCM Consensus Conference publications and other clinical definitions of sepsis could have allowed such large numbers of patients to be enrolled in sepsis studies, whose host-inflammatory responses to infection were unable to benefit from the test compound that their treatment effects were lost within a nonspecific clinical trial population. The true target population for each sepsis drug, then, could be diluted to into invisibility by the overwhelming numbers of nonresponders enrolled. As a result, potentially life-saving drugs for sepsis and septic shock may not have received a fair chance to prove their efficacy but still were deemed ineffective because they were evaluated in what were otherwise thought to be well-designed clinical trials.

Accordingly, there is a need in the art for improved methods of evaluating clinical trial data and identifying subjects suitable for a particular clinical trial as well as identifying subjects, in a clinical setting, that will respond or gain benefit from a therapeutic agent.

### SUMMARY OF THE INVENTION

The present invention is a system composed of (a) a non-transitory computer-readable medium having stored thereon a program that includes (i) a patient data input module configured to receive data regarding a patient from a client terminal via a network, the data including one or more baseline parameters of a patient with a disease or condition, wherein said baseline parameters include one or more demographic variables, physiologic variables, gene expression profiles or results of hospital laboratory tests, and (ii) an outcome prediction module configured to generate from the baseline parameters a systemic mediator-associated response test (SMART) profile for the patient, and predict an outcome of a therapeutic agent for treatment of the patient's disease or condition based at least on the SMART profile and on historical data regarding outcomes of the therapeutic agent administered to a plurality of patients; and (b) a processor configured to execute the program and to cause the predicted outcome to be displayed on a display.

The present invention is also a system composed of a computer system configured to communicate with a client terminal via a network, the computer system comprising a processor configured to (i) predict an outcome of a therapeutic agent for the treatment of a patient having a disease or condition, said outcome being based at least on patient data and on historical data regarding outcome of the therapeutic agent administered to a plurality of patients, the patient data including baseline parameters comprising one or more demographic variables, physiologic variables, gene expression profiles or results of hospital laboratory tests; and (ii) communicate the predicted outcome to the client terminal via the network for display on the client terminal.

In some embodiments of the disclosed systems, the patient is in or being considered for a clinical trial and the baseline parameters are prerandomization baseline parameters. In other embodiments, the outcome prediction module is configured to communicate the predicted outcome of each of the therapeutic agent to the client terminal via the network. In further embodiments, the outcome prediction module is configured to (i) compare the received data regarding the patient with corresponding data regarding the plurality of patients, (ii) based on the comparison, determine a subset of the plurality of patients with which the patient most closely correlates, and (iii) determine the predicted outcome of the therapeutic agent using the outcomes for the subset of the plurality of patients. In yet other embodiments, the outcome prediction module is configured to predict the outcome using at least one of multivariate regression analysis, univariate analysis, advanced regression analysis, fully saturated regression analysis, stepwise regression analysis, and least angle regression analysis.

### DETAILED DESCRIPTION OF THE INVENTION

Systems and methods for prognosticating clinical events in sepsis and other diseases or conditions have now been developed. The systems and methods involve the generation of a systemic mediator-associated response test (SMART) model for a particular therapeutic agent or patient using baseline data and objectively identifying patients who are likely to positively respond to the particular therapeutic agent. By way of illustration, the studies described herein analyzed results of sepsis clinical trials that used consensus definitions as entry criteria, in which the study test molecule failed to reduce septic mortality. The results of this study illustrated SMART's ability to identify objectively, from prerandomization baseline data, patients within failed clinical trials among whom novel treatments reduce septic mortality. SMART also predicted which sepsis drugs may not be beneficial. Specifically, SMART models uncovered cohorts of septic patients wherein E5, TNFMAb, and IL-1ra improved survival significantly. Furthermore, the SMART models built on the NORASEPT (North American Sepsis Trial) database, and efficacy of the TNFMAb study drug, were validated prospectively in NORASEPT II. Conversely, the failure of platelet-activation factor acetylhydrolase (PAF-AH) to lower septic mortality, and its possible adverse effects, was predicted early in the COMPASS (COntrolled Mortality trial of human Platelet-activating factor Acetylhydrolase for treatment of Severe Sepsis) study database by SMART. However, even in maximally steroid-responsive SMART cohorts, hydrocortisone did not improve septic shock survival, neither in CORTICUS (Corticosteorid Treatment of Septic Shock) overall nor in corticotrophin non-responders. These results were achieved using clinical trial databases that were uncontrolled for optimal statistical modeling, and through analyzing only ordinary bedside observations and standard hospital laboratory tests, without the potentially valuable contributions of circulating levels of inflammatory response mediators or other sepsis biomarkers.

Treatments for a number of conditions have failed to reach their full potential because early subclinical identification of appropriate patients to participate in clinical efficacy studies has proven most difficult. Physiologic scoring systems which are used by physicians to predict ‘mortality in a patient have generally proven insufficient in predicting the onset of certain conditions subclinically. Accordingly, in one embodiment of the invention, SMART profiles are used in systems and methods for analyzing clinical trial results to predict outcomes and improve efficacy of a therapeutic agent. This methodology involves (a) obtaining data from the patient with the disease or condition, wherein said patient data includes baseline parameters comprising one or more demographic variables, physiologic variables, gene expression profiles and/or results of hospital laboratory tests; generating, from the baseline parameters, a systemic mediator-associated response test (SMART) profile for the patient; comparing the SMART profile with corresponding historical data regarding outcomes of the therapeutic agent administered to a plurality of patients; based on the comparison, determining a subset of the plurality of patients with which the patient most closely correlates; and predicting the outcome of the therapeutic agent using the outcomes for the subset of the plurality of patients. The predicted outcome can then be used by a clinician or physician to evaluate whether the patient will respond positively or receive benefit from treatment with the therapeutic agent and/or be assess whether a patient should be included in or removed from a clinical trial.

Clinical trials that can be analyzed using the method of this invention include clinical trials for therapeutic agents of use in the prevention or treatment of diseases or conditions of the central nervous system (CNS) or peripheral nervous system and disorders of the peripheral organs. Disorders of the CNS include stroke, aging, neurodegenerative conditions, such as Alzheimer's disease, Parkinson's disease, aneurysm, migraine and other vascular headaches, HIV-dementia, cancer and the like. Disorders of the peripheral nervous system include diabetic peripheral neuropathy and traumatic nerve damage. Peripheral organ disease includes atherosclerosis, cancer, chronic obstructive pulmonary disease (COPD), pancreatitis, pulmonary fibrosis, angioplasty, trauma, ischemic bowel disease, lupus, renal hypertension, autoimmune conditions, such as systemic lupus (erythematosus), multiple sclerosis and the like; and inflammatory conditions, such as inflammatory bowel disease, Crohn's disease, ulcerative colitis, rheumatoid arthritis, septic shock, septicemia, and the like. Some disease conditions may be classified as, for example, both autoimmune and inflammatory conditions, such as multiple sclerosis, ulcerative colitis, and the like. In certain embodiments, the disease or condition is an inflammatory disease or condition such as inflammatory bowel disease, Crohn's disease, ulcerative colitis, rheumatoid arthritis, and systemic inflammatory conditions such as septic shock, septicemia, and the like.

The development of systemic inflammatory conditions represents a significant portion of the morbidity and mortality incidence which occur in the intensive care unit (ICU). The term “systemic inflammatory conditions” is used herein to describe conditions which result in a host response manifested by increased capillary permeability, organ failure, and death. Examples of systemic inflammatory conditions include, but are not limited to, ARDS, SIRS, sepsis, MODS, single organ dysfunction, shock, transplant rejection, cancer and trauma. Systemic inflammatory conditions such as ARDS, SIRS and MODS are responsible for more than 70% of the ventilator days spent on the ICU. In addition, ARDS, SIRS, sepsis and MODS are primary causes of death following surgery in surgical ICU patients, thus placing a heavy burden on the health care system. Generally, systemic inflammatory conditions do not develop in healthy individuals but rather in patients with preexisting severe disease or in persons who have suffered catastrophic acute illness or trauma. Patients at greatest risk of dying of a systemic inflammatory condition are the elderly; those receiving immunosuppressive drugs; and those with malignancies, cirrhosis, asplenia, or multiple underlying disorders (Bone (1991) Annals of Internal Medicine 115:457-469).

Identifying subjects likely to respond positively to a therapeutic agent and optimizing treatment for patients having one or more of the above diseases or conditions would be especially useful to clinicians. Once a patient is predicted to favorably respond to a therapeutic agent based on comparison of his or her SMART profile to a control SMART profile established in the clinical population that responded to treatment, the physician would employ their experience and judgment in determining the appropriate mode and timing of treatment.

Based upon the results presented herein, the system and method of the present invention can be used to identify treatments that could be used successfully to treat patients with one or more diseases or conditions, in particular patients with an inflammatory condition, e.g., a systemic inflammatory condition such as severe sepsis. In this respect, systems and methods are also provided for matching patients with other novel treatments based upon comparison of SMART profiles for the patient and established control profiles for effective treatments or new treatments with predetermined mechanisms of action (e.g., inhibiting tumor necrosis factor, inhibiting endotoxin activity, inhibiting interleukin-1 receptor, or degrading platelet-activating factor and oxidized phospholipids). By matching patients whose physiological responses to a disease or condition is matched with a mechanism of action of a therapeutic agent, effective treatments for patients can be selected. The optimization of treatment for patient populations with the present system and method is an improvement on current methods of clinical trial data analysis and increases the likelihood that efficacy will be shown in the clinical trial. In the system and method of the invention, patient data including baseline parameters is entered into a patient input model of a computer, and a SMART profile regarding these baseline parameters is generated by an output prediction module. Using statistical tests executed by the output prediction module, the patient SMART profile is compared to established control profiles that include corresponding data from a plurality of patients for which treatment with the therapeutic agent of interest is effective and/or has the same predetermined mechanism of action. Based upon these comparisons, a predicted outcome for the therapeutic agent in the treatment of the patient's disease or condition can be made. In certain embodiments, at least one independent variable of the patient's SMART profile matches, overlaps or correlates with an independent variable of a control profile. An overlap, e.g., at least one, two, three, four, five, six, seven or more independent variables in common, or alternatively, 50%, 60%, 70%, 80%, 90% or 100% overlap in independent variables of a control SMART profile and a patient's SMART profile indicates that the patient is likely to respond to treatment with the therapeutic agent. In this respect, appropriate patient populations for testing of new drugs in development can be selected via matching of patients with treatments based upon SMART profiles. By “appropriate patient population” it is meant patient who meet the clinical entry criteria of a study for a new drug, and whose SMART profile matches or most closely correlates with data from a subset of patients that benefited from treatment with the new drug or drug with a similar mechanism of action, such that the patient will likely respond positively to the new drug if randomized to it.

For purposes of this invention, a “control profile,” “control SMART profile” or “clinical population profile” can be generated from a database containing mean values (e.g., of independent variables) for historical data from a plurality of patients being treated for a particular disease or condition as described herein. Upon comparing the patient SMART profile with the control profile, a subset of patients within control profile whose data most closely correlates with the patient SMART profile is identified via statistical analyses. Ideally, this subset of patients received benefit from treatment with the therapeutic agent such that the patient to be treated would also receive benefit from treatment with the therapeutic agent.

In other embodiments, a “control profile” can be generated from data obtained from the same patient to compare and monitor changes in the patient parameters over time.

Patient and control SMART profiles of the present invention are generated from one or more baseline parameters or more particularly prerandomized baseline parameters. Patient parameters, for purposes of this invention, may include selected demographic variables, selected physiologic variables, gene expression profiles and/or results from selected standard hospital laboratory tests.

Exemplary demographic variables which may be selected for inclusion in a SMART profile include, but are not limited to, age, sex, race, comorbidities such as alcohol abuse, cirrhosis, HIV, dialysis, neutropenia, COPD, solid tumors, hematologic malignancies, chronic renal failure and the admitting service, i.e., surgery or medicine, and trauma.

Examples of physiologic variables which may be selected for inclusion in a SMART profile include, but are not limited to, physical examination, vital signs, hemodynamic measurements and calculations, clinical laboratory tests, concentrations of acute inflammatory response mediators, and endotoxin levels. More specifically, physiologic variables selected may include height, weight, temperature, MAP, heart rate, diastolic blood pressure, systolic blood pressure, mechanical ventilation, respiratory rate, pressure support, PEEP, SVR, cardiac index and/or PCWP of the patient. In addition, complete blood count, platelet count, prothrombin time, partial thromboplastin time, fibrin degradation products and D-dimer, serum creatinine, lactic acid bilirubin, AST, ALT, and/or GGT can be measured. Heart rate, respiratory rate, blood pressure and urine output can also be monitored. A full hemodynamic profile can also recorded in patients with pulmonary artery catheters and arterial blood gases are performed in patients on ventilators. Chest X-rays and bacterial cultures can also performed as clinically indicated. Examples of inflammatory response mediators which can be determined from a biological sample obtained from the patient include, but are not limited to, prostaglandin 6-keto F1α (PGI) (the stable metabolite of prostacyclin), thromboxane B₂ (TxB) (the stable metabolite of thromboxane A₂), leukotrienes B₄, C₄, D₄ and E₄, interleukin-6, interleukin-8, interleukin-1β, tumor necrosis factor, neutrophil elastase, complement components C3 and C5a, platelet activating factor, nitric oxide metabolites and endotoxin levels.

Examples of gene expression profiles of use in this invention include, but are not limited to, upregulation and/or downregulation of expression of particular genes, alterations in protein levels or modification, or changes at the genomic level (such as mutation, methylation, etc). Gene expression profiles can include, but is not limited to, the expression of one or more protein kinases (e.g., creatine kinase), growth factors (e.g., insulin-like growth factor, transforming growth factor β), hormones (e.g., growth hormone 2, hepatoma-derived growth factor), enzymes (e.g., nitric oxide synthase, superoxide dismutase, phospholipase, lysozyme, matrix metalloproteinase (MMP) 12, MMP9, MMP1, MMP3, aldolase B, esterase D), chemokines or cytokines (e.g., IL-6, IL-8, IL-9), receptors (e.g., IL-1RA), transcription factors (e.g., transcription factor IIIa), zinc finger proteins (e.g., zinc finger protein 91), structural proteins (e.g., collagen), inflammatory mediators, cell cycle regulators, HLA or immune function genes, antimicrobial genes, extracellular matrix and remodeling genes, carbohydrate metabolism genes, fatty acid metabolism genes, etc.

Exemplary hospital laboratory tests considered standard by those skilled in the art which may be selected for inclusion in a SMART profile include, but are not limited to, levels of albumin, alkaline phosphatase, ALT, AST, BUN, calcium, cholesterol, creatinine, GGT, glucose, hematocrit, hemoglobin, MCH, MCV, MCHC, phosphorus, platelet count, potassium, total protein, PT, PTT, RBC, sodium, total bilirubin, triglycerides, uric acid, WBCL, base deficit, pH, PaO₂, SaO₂, FiO₂, chloride, and lactic acid.

Some or all of these patient parameters are preferably determined at baseline (before drug treatment, drug intervention or before randomization to a clinical trial, i.e., prerandomization), and daily thereafter where applicable, and are entered into a database and a SMART profile comprising one or more of the patient parameters is generated from the database. As one of skill in the art will appreciate from this disclosure, as other additional patient parameters are identified as independent variables, they can also be incorporated into the database and as part of the SMART profile. Similarly, as SMART profiles are generated for more patients and additional data are collected for these parameters, it may be found that some parameters in this list of examples are less predictive than others. Those parameters identified as less predictive in a larger patient population need not be included in all SMART profiles.

Examples of biological samples from which some of these physiologic parameters are determined include, but are not limited to, blood, plasma, serum, urine, bronchioalveolar lavage, sputum, and cerebrospinal fluid.

As will be understood by those of skill in the art upon reading this disclosure, SMART profiles can be generated from all of the patient parameters discussed supra. Alternatively, SMART profiles can be based upon only a portion of the patient parameters. Since the patient parameters for each patient, as well as the control profiles or clinical population profile, are stored in a database, various SMART profiles comprising different patient parameters can be generated for a single patient and compared to an established control profile (i.e., historical data) comprising the same parameters. The ability of these various profiles to be predictive can then be determined via statistical tests executed on a computer.

Comparisons between the patient SMART profile and historical data from a plurality of patients can be based upon continuous, normally distributed variables using one or more statistical analyses including, but not limited to, multivariate regression analysis, univariate analysis, advanced regression analysis, fully saturated regression analysis, stepwise regression analysis, and least angle regression analysis. When appropriate, statistical comparisons between subgroups are made using the t-test or the chi-squared equation for categorical variables. The results of such analyses provide profiles comprising independent variables for patients who respond positively to a therapeutic agent, i.e., the subject has an improvement or amelioration in one or more signs or symptoms of the disease or condition being treated.

Predicted outcomes are provided as an output, e.g., on a display such as a monitor, screen, or print out, which are used by a clinician or physician to assess whether a patient will respond positively to the therapeutic agent. Thus, the SMART methodology can supplement clinical entry criteria for studies of antibiotics, cancer treatments, and transplant regimens, among others, as well as new drugs for sepsis, acute organ failure, and other systemic inflammatory conditions. SMART profiles ensure that the study drug receives a reasonable chance to demonstrate its efficacy in the conditions under treatment. After SMART profiling is used to demonstrate a drug's efficacy, SMART profiles can then be applied at the bedside to identify individual patients for whom the drug in question is beneficial. Using SMART, the host inflammatory response of individuals can now be matched to the biopharmacologic properties of a drug. This system and method is therefore a way to enhance the likelihood that clinical efficacy will be demonstrated in clinical trials, in particular phase II, III, and IV clinical trials.

Computer System. The systems and methods disclosed herein can be implemented using one or more computer systems, which are also referred to herein as digital data processing systems. Various exemplary embodiments of computer systems are described in, e.g., U.S. Pat. No. 8,036,912, incorporated by reference in its entirety.

The computer system of this invention can include one or more processors which can control the operation of the computer system. The processor(s) can include any type of microprocessor or central processing unit (CPU), including programmable general-purpose or special-purpose microprocessors and/or any one of a variety of proprietary or commercially available single or multi-processor systems. The computer system can also include one or more memories, which can provide temporary storage for code to be executed by the processor(s) or for data acquired from one or more users, storage devices, and/or databases. The memory can include read-only memory (ROM), flash memory, one or more varieties of random access memory (RAM) (e.g., static RAM (SRAM), dynamic RAM (DRAM), or synchronous DRAM (SDRAM)), and/or a combination of memory technologies.

The various elements of the computer system can be coupled to a bus system. The bus system can represent any one or more separate physical buses, communication lines/interfaces, and/or multi-drop or point-to-point connections, connected by appropriate bridges, adapters, and/or controllers. The computer system can also include one or more network interface(s), one or more input/output (I/O) interface(s), and one or more storage device(s).

The network interface(s) can enable the computer system to communicate with remote devices, e.g., other computer systems, over a network, and can be, for non-limiting example, remote desktop connection interfaces, Ethernet adapters, and/or other local area network (LAN) adapters. The I/O interface(s) can include one or more interface components to connect the computer system with other electronic equipment. For non-limiting example, the I/O interface(s) can include high speed data ports, such as universal serial bus (USB) ports, 1394 ports, etc. Additionally, the computer system can be accessible to a human user, and thus the I/O interface(s) can include displays, speakers, keyboards, pointing devices, and/or various other video, audio, or alphanumeric interfaces. The storage device(s) can include any conventional medium for storing data in a non-volatile and/or non-transient manner. The storage device(s) can thus hold data and/or instructions in a persistent state, i.e., the value is retained despite interruption of power to the computer system. The storage device(s) can include one or more hard disk drives, flash drives, USB drives, optical drives, various media cards, and/or any combination thereof and can be directly connected to the computer system or remotely connected thereto, such as over a network. The elements of the system can be some or all of the elements of a single physical machine. In addition, not all of the elements need to be located on or in the same physical machine. Exemplary computer systems include conventional desktop computers, workstations, minicomputers, laptop computers, tablet computers, personal digital assistants (PDAs), mobile phones, and the like.

The computer system can include a web browser for retrieving web pages or other markup language streams, presenting those pages and/or streams (visually, aurally, or otherwise), executing scripts, controls and other code on those pages/streams, accepting user input with respect to those pages/streams (e.g., for purposes of completing input fields), issuing Hypertext Transfer Protocol (HTTP) requests with respect to those pages/streams or otherwise (e.g., for submitting to a server information from the completed input fields), and so forth. The web pages or other markup language can be in HyperText Markup Language (HTML) or other conventional forms, including embedded Extensible Markup Language (XML), scripts, controls, and so forth. The computer system can also include a web server for generating and/or delivering the web pages to client computer systems.

While some embodiments are described herein in the context of web pages, a person skilled in the art will appreciate that in other embodiments, one or more of the described functions can be performed without the use of web pages and/or by other than web browser software. A computer system can also include any of a variety of other software and/or hardware components, including by way of non-limiting example, operating systems and database management systems with or without access to a network.

Outcome Prediction System. The system of this invention can include a plurality of modules, discussed further below, which can each be implemented using one or more digital data processing systems of the type described above, and in particular using one or more web pages which can be viewed, manipulated, and/or interacted with using such digital data processing systems. The system can thus be implemented on a single computer system, or can be distributed across a plurality of computer systems. The system also includes a plurality of databases, which can be stored on and accessed by computer systems. It will be appreciated that any of the modules or databases disclosed herein can be subdivided or can be combined with other modules or databases. The system can be a computer-based system configured similar to embodiments described in U.S. Pat. No. 8,036,912.

Any of a variety of parties can access, interact with, control, etc. the system from any of a variety of locations. For non-limiting example, the system can be accessible over a network (e.g., over the Internet via cloud computing) from any number of client stations in any number of locations such as a medical facility (e.g., a hospital, a medical clinic, a doctor's office, a mobile medical facility, etc.), a home base (e.g., a patient's home or office, a doctor's home or office, etc.), a mobile location, and so forth. The client station(s) can access the system through a wired and/or wireless connection to the network. The system can allow the client station(s) to upload data to the system over the network and download data from the system over the network. In an exemplary embodiment, at least some of the client terminal(s) can access the system wirelessly, e.g., through Wi-Fi connection(s), 3G connections, 4G connections, etc., which can facilitate accessibility of the system from almost any location in the world. A medical facility can include client stations in the form of a tablet and a computer touch screen, a home base can include client stations in the form of a mobile phone having a touch screen and a desktop computer, and a mobile location can include client stations in the form of a tablet and a mobile phone, but the medical facility, the home base, and the mobile location can include any number and any type of client stations. In an exemplary embodiment, the system can be accessible by a client terminal via a web address and/or a client application (generally referred to as an “app”).

A person skilled in the art will appreciate that the system can include security features such that the aspects of the system available to any particular user can be determined based on the identity of the user and/or the location from which the user is accessing the system. To that end, each user can have a unique username, password, and/or other security credentials to facilitate access to the system. The received security parameter information can be checked against a database of authorized users to determine whether the user is authorized and to what extent the user is permitted to interact with the system, view information stored in the system, and so forth. Exemplary, non-limiting examples of parties who can be permitted to access the system include patients, potential patients, surgical technicians, nurses, general medical practitioners, and medical students.

The system can include a patient data input module, a historical data input module, and an outcome prediction module. Any of the patient data input module, the historical data input module, and the outcome prediction module can be used independently from one another and can be used in combination with any one or more other modules. The system can also include a patient data database configured to be accessible by the patient data input module and to store patient data, a historical data database configured to be accessible by the historical data input module and to store historical data, and an educational information database configured to be accessible by the outcome prediction module and to store educational data. Each of the databases can include any number of component databases, e.g., one, two, three, etc., the same or different from any of the other databases. A person skilled in the art will appreciate that any of the databases and any of their various component databases (if any), can be subdivided or can be combined with other databases. Any portion of any of the databases can be configured to be accessed, e.g., read from and/or written to, by any one or more of the modules and any additional module(s) (if any).

Users of the system can include patients and medical practitioners involved with treating one or more of the patients. In some embodiments, the system can be accessible by users other than patients and medical practitioners, such as by medical students, family members of patients, etc. Different users can have access to different portions of the system, as mentioned above regarding security features. As a non-limiting example, the system can be configured to allow patients to access the patient data input module, to allow medical administrators to access only the historical data input module, and to allow medical professionals and medical students to access all of the modules. A user can have access to only a portion of a module, e.g., to only a subset of component modules within any one or more of the modules.

Generally, the system can be configured to allow patient 400 to be input via the patient data input module and historical data to be input via the historical data input module. The outcome prediction module can be configured to analyze the input patient data and the input historical data so as to output one or more predicted outcomes of one or more therapeutic agents.

Patient Data Input Module. The patient data input module can generally provide users of the system with an interface for entering data regarding patients and submitting the data to the system. The submitted patient data can then be used by the outcome prediction module to predict treatment outcomes for the patient.

As mentioned above, the patient data input module can be configured to read information from and/or write information to the patient data database. Thus, the patient data input module can be configured to write submitted patient data to the patient data database. The patient data can be organized in any way in the patient data database and/or in one or more other storage areas accessible by the system. In an exemplary embodiment, patient data can be stored in a table in the patient data database such that each patient has his/her own row or column of data populated with data related to that patient. However, as will be appreciated by a person skilled in the art, patient data can be stored in any way.

The patient data input module can be configured to automatically gather patient data and/or can be configured to receive manually input patient data, e.g., receive patient data submitted thereto. In an exemplary embodiment, the patient data input module can be configured to automatically gather data and to manually receive data, thereby maximizing an amount of data that the system can consider in evaluating treatment outcomes for patients. By automatically gathering patient data, the patient data input module can help ensure that the most recent and comprehensive patient data is available for analysis by the system, help account for accidental omission of manual patient data entry to the system, and/or help ensure that accurate patient data is received by the system. By allowing manual patient data entry, the patient data input module can help allow data to be input and considered that is more current than data available in a storage unit automatically accessible by the patient data input module and help allow input and consideration of data not accessible through automatic data gathering.

The patient data input module can be configured to automatically gather patient data in a variety of ways. In an exemplary embodiment, the patient data input module can be configured to transmit a request for patient data information to one or more storage units storing patient data, e.g., patient medical records stored at a medical facility such as a hospital, doctor's office, clinic, etc., patient insurance information stored at a medical facility, insurance carrier office, etc., and other types of patient data. In response to the request, the one or more storage units can transmit the requested patient data to the patient input data module. To help ensure confidentiality of patient data, any one or more security measures can be taken in requesting and/or transmitting the data, such as encrypting the patient data request, encrypting the transmitted patient data, authenticating the patient data input module via one or more authentication mechanisms (e.g., passwords, keys, etc.), temporarily storing the patient data for a single user session (e.g., storing the patient data until the user who requested automatic gathering of the patient data logs off the system), etc.

The patient data input module can be configured to automatically gather patient data at predetermined time intervals and/or on demand (e.g., by user request after user login to the system over the network). The predetermined time intervals can be preset, and can be any time interval, e.g., every thirty days, every six months, every day, etc. The predetermined time intervals can be the same for all patients or different for different patients. For non-limiting example, the patient data input module can be configured to automatically gather patient data from certain medical facilities (e.g., hospitals) more frequently than other medical facilities (e.g., clinics) because patients visiting the certain medical facilities which provide specialized medical care can be considered to be more likely candidates for treatment/clinical trials than patients visiting the other medical facilities which provide more generalized medical care, and hence more likely users of the system.

The patient data input module can be configured to receive patient data manually submitted thereto in a variety of ways. In one embodiment, the patient data input module can be implemented using one or more web pages which are configured to receive user input and present information to a user. In an exemplary embodiment, both patients and medical practitioners can access at least a portion of the patient data input module. In an exemplary embodiment, the patient data input module can be accessed by users via a web interface, e.g., by connecting to the Internet via a client terminal and accessing a specific web address, by launching an app on a client terminal that accesses the system, etc. As mentioned above, the users can wirelessly access the system, including the patient data input module, and can submit the data to the system via the web, e.g., by clicking on a “submit” button on a web page.

The patient data input module can be configured to receive a variety of different types of data regarding a patient. Non-limiting examples of patient data that can be received (automatically and/or manually) by the patient data input module include identification data, demographic data, physiologic data, gene expression data and results of hospital laboratory tests. Non-limiting examples of identification data include a unique patient identifier (e.g., a name, a social security number, an insurance identification code, a system logon name, a hospital identification code), a geographic location of the patient (e.g., country, zip code, etc.), etc. Examples of demographic data, physiologic data, gene expression data and results of hospital laboratory tests are described herein.

The web interface configured to allow users to access the patient data input can have a variety of configurations. Users who access the system may or may not be the same person as the patient for whom the user is inputting patient data and/or requesting predicted outcomes of treatment. The web interface can be configured to be displayed on a client terminal, as can any of the various web interfaces described herein.

The patient data web interface can identify one or more therapeutic agents for which the input patient data can be analyzed, as discussed further below, to predict an outcome for the patient if the patient receives treatment with the one or more therapeutic agents. The system can thus allow predicted outcomes for multiple therapeutic agents to be directly compared against one another, which can help allow users such as patients and medical practitioners to make better, more informed decisions as to which therapeutic agent may be most effective for a particular patient.

The patient data web interface includes patient data entry fields including, e.g., a height field (asking for height to be entered in inches, although entry in any length unit can be requested or provided on any patient data web interface), a weight field (asking for weight to be entered in pounds, although entry in any weight unit can be requested or provided on any patient data web interface), a gender field, an age field (asking for age to be entered in years, although entry in any time unit can be requested or provided on any patient data web interface), an insurance status field, an employment status field, a comorbidity or other medical condition field, other medication field, etc. Any one or more of the fields of the patient data web interface can allow data entry thereto in any number of ways, as will be appreciated by a person skilled in the art, such as by text box, radio button, push button, drop-down menu, list box, check box, cycle button, etc. Further, any one or more of the fields can be configured to be exclusively automatically populated with patient data (such as if the patient data input module is configured to automatically retrieve patient data from the patient data database (e.g., retrieve patient data previously entered by the user into the system and saved in the patient data database, retrieve patient data from one or more remote storage units, retrieve patient data from the patient data database previously automatically retrieved by the patient data input module according to a predetermined data retrieval schedule, etc.)), exclusively manually populated with patient data (such as if the patient data input module is not configured to automatically retrieve patient data or to conserve processing resources), or to be manually or automatically populated.

The patient data web interface can be configured to allow user access to one or more educational materials stored in the educational information database in any number of ways. For example, the patient data web interface can be configured to allow a user to select, e.g., scroll over, click on an hourglass icon thereon, etc., one or more of information boxes regarding one or more therapeutic agents. The information boxes each identify its associated therapeutic agent by name and can provide additional information such as side effects, etc. In response to the user selecting one or more of the information boxes, the patient data input module can be configured to retrieve one or more educational materials related to the selected therapeutic agent from the educational information database and cause the educational materials and/or links thereto to be displayed on the patient data web interface directly, on the patient data web interface in a pop-up box, on another web interface, etc. The system can therefore be configured to help educate the user about different therapeutic options. Non-limiting examples of educational materials include links to informational web pages stored in the system (e.g., in the educational information database), links to third party educational websites, lists of or links to journal articles or books that are stored in the system (e.g., in the educational information database), links to and/or copies of product brochures (e.g., brochures stored electronically in the system), etc.

Fields having numerical inputs can be configured to accept data in one or more units and be configured to automatically convert the data input in one of the units to the other units. If input values are outside of a range for the system (e.g., a height entered beyond a predetermined threshold indicating an upper height limit which adult humans do not presently exceed, etc.), the system can be configured to display an error message indicating an appropriate corrective action, e.g., entering a new number, removing a decimal point, etc. For non-limiting example, weight entered in pounds in a pounds data field in the weight field can be automatically converted to weight in kilograms, and a kilograms data field in the weight field can be automatically populated. For another non-limiting example, height entered in feet and inches in feet and inches data fields in the height field can be automatically converted to height in centimeters, and a centimeters data field in the height field can be automatically populated.

Any of the fields in any of the areas can be configured to accept only predetermined answers thereto, e.g., not be a text box in which a user can enter any text. The input data can therefore be one of a plurality of possible user selections, thereby allowing the system to accurately identify and analyze input data. In an illustrated embodiment, the race field, the ethnicity field, insurance field and employment field can allow predetermined answers by providing drop-down menus of possible answers that can be selected by a user.

The patient data web interface can include a submitter configured to allow user inputs to the areas to be submitted to the system. The submitter can include a clickable button marked “GO,” but any text (e.g., “OK,” “submit,” “calculate,” etc.) can be on the button. Further, the submitter need not be a clickable button, as will be appreciated by a person skilled in the art, but can be any submission mechanism, such as a check box, an audio command receiver, a user movement detector for a touch pad or touch screen, etc.

In some embodiments, a user's selection of a predetermined answer, e.g., “yes” from “yes/no,” inputting a check box next to a particular medical condition, etc., can be configured to trigger display and/or activation of one or more additional fields for the user to complete. For non-limiting example, selecting a particular medical condition as being an affliction of a patient can trigger display and/or activation of a medication field for that particular medical condition in which the user can indicate any medications being taken to treat the particular medical condition. For another non-limiting example, indicating that a patient is currently taking a medication can trigger display and/or activation of a time field in which the user is prompted to enter how long (e.g., number of days, weeks, months, years, etc.) the patient has been taking the medication. Similarly, in some embodiments, a user's entry of certain text in a text box can trigger display and/or activation of one or more additional fields for the user to complete. For non-limiting example, a user entering an age in a predetermined range can trigger a first set of one or more questions appropriate for that age range, and a user entering an age in another predetermined range can trigger a second, different set of one or more questions appropriate for that age range.

In some embodiments, a user's selection of a predetermined answer or entry of certain text in a text box can cause predetermined information to be displayed on the patient data web interface. For non-limiting example, user entry of a patient's height in one unit, e.g., feet/inches, can trigger display of the patient's height in one or more other height units, e.g., centimeters, meters, etc.

Historical Data Input Module. The historical data input module can generally provide users of the system with an interface for entering data regarding treatment outcomes or clinical trial results and submitting the data to the system. The submitted patient data can then be used by the outcome prediction module to predict treatment outcomes for a patient.

As mentioned above, the historical data input module can be configured to read information from and/or write information to the historical data database. Thus, the historical data input module can be configured to write submitted historical data to the historical data database. The historical data can be organized in any way in the historical data database and/or in one or more other storage areas accessible by the system.

The historical data input module can be configured to automatically gather historical data and/or can be configured to receive manually input historical data, e.g., receive historical data submitted thereto. In an exemplary embodiment, the historical data input module can be configured to automatically gather data and to manually receive data, thereby maximizing an amount of data that the system can consider in evaluating treatment outcomes for patients. By automatically gathering patient data, the historical data input module can help ensure that the most recent and comprehensive data is available for analysis by the system, help account for accidental omission of manual historical data entry to the system, and/or help ensure that accurate historical data is received by the system. By allowing manual historical data entry, the historical data input module can help allow data to be input and considered that is more current than data available in a storage unit automatically accessible by the historical data input module and help allow input and consideration of data not accessible through automatic data gathering.

The historical data input module can be configured to automatically gather historical data in a variety of ways. In an exemplary embodiment, the historical data input module can be configured to transmit a request for historical data information to one or more storage units storing historical data, e.g., clinical trial data stored at a medical facility such as a hospital, doctor's office, clinic, etc., insurance reimbursement information stored at a medical facility, insurance carrier office, Thomson Reuters MarketScan®, etc., joint registry data, Premier hospital database, MedAssets data, Ingenix/I3 data, Gesinger data, MedPar data, HCUP data, national health care databases (e.g., databases indexed by the International Society for Pharmacoeconomics and Outcomes Research (ISPOR) such as the National Institute of Statistics and Censuses database of Argentina and the National Health and Wellness Survey database of France), and other types of historical data. Relevant historical data (e.g., data related to specific clinical trials) can be identified in and retrieved from stored historical data, e.g., stored in an external database, in a variety of ways, as will be appreciated by a person skilled in the art, such as by identifying and retrieving data associated with a specific International Classification of Diseases (ICD) code (e.g., a specific ICD-9 code) or a specific Current Procedural Terminology (CPT) code. In response to the request, the one or more storage units can transmit the requested historical data to the historical data input module. To help ensure confidentiality of historical data, any one or more security measures can be taken in requesting and/or transmitting the data, such as encrypting the historical data request, encrypting the transmitted historical data, authenticating the historical data input module via one or more authentication mechanisms (e.g., passwords, keys, etc.), storing data in the historical data database without any specific patient identifiers (e.g., data not being associated with a particular patient name, patient identification code, etc.), etc.

The historical data input module can be configured to automatically gather historical data at predetermined time intervals and/or on demand (e.g., by user request after user login to the system over the network). The predetermined time intervals can be preset, and can be any time interval, e.g., every thirty days, every six months, every day, etc. The predetermined time intervals can be the same for all types of historical data or different for different types of historical data. For non-limiting example, the historical data input module can be configured to automatically gather historical data from certain medical facilities (e.g., hospitals) more frequently than other medical facilities (e.g., clinics) because patients visiting the certain medical facilities which provide specialized medical care can be considered to be more likely candidates for treatment than patients visiting the other medical facilities which provide more generalized medical care.

The historical data input module can be configured to receive historical data manually submitted thereto in a variety of ways. In one embodiment, the historical data input module can be implemented using one or more web pages which are configured to receive user input and present information to a user. In an exemplary embodiment, medical practitioners but not patients can access at least a portion of the historical data input module. In an exemplary embodiment, the historical data input module can be accessed by users via a web interface, e.g., by connecting to the Internet via a client terminal and accessing a specific web address, by launching an app on a client terminal that accesses the system, etc. As mentioned above, the users can wirelessly access the system, including the historical data input module, and can submit the data to the system via the web, e.g., by clicking on a “submit” button on a web page. For non-limiting example, historical data can be manually entered by one or more medical professionals performing a check-up on a patient previously having treated with a therapeutic agent of interest thereon by entering the patient's check-up data into a computer system maintaining patient data. The computer system can be the system or another system configured to communicate with the system. The patient's check-up data can thus be stored in the historical data database.

The historical data input module can be configured to receive a variety of different types of historical data regarding treatment with a therapeutic agent. Non-limiting examples of historical data that can be received (automatically and/or manually) by the historical data input module include a change in one or more medical conditions for a patient having received treatment with the therapeutic agent, and a genetic indicator of a patient having received treatment with the therapeutic agent.

The web interface configured to allow users to access the historical data input module can have a variety of configurations and can generally be configured similar to any of the web interfaces discussed above for the patient data input module.

Outcome Prediction Module. The outcome prediction module can generally provide users of the system with an interface for receiving one or more predicted outcomes of treatment of a patient with a therapeutic agent. Various predictive analyses can be performed by the outcome prediction module, and various models can be used in performing the predictive analyses.

In general, predictive models require good databases, e.g., collections of reliable data. Non-limiting examples of characteristics of a good database for the outcome prediction module include longitudinal patient data and large data sets. How “large” the data set should be to ensure, sound predictions depends on the strength of correlations. Longitudinal data generally includes data both before and after treatment with a sufficient number of points in between, e.g., a statistically significant number of points in between, and includes inputs and outputs in sufficient numbers, e.g., statistically significant numbers, to determine correlations. Exemplary data include the baseline parameters (e.g., prerandomized baseline parameters) described herein.

With access to a good database, which can include one or more separate databases, the outcome prediction module can be configured to identify if correlations can be determined between potential inputs and outputs. In an exemplary embodiment, the outcome prediction module can be configured to perform this identification without bias, in an agnostic way to let the data from the database(s) reveal what the correlations are.

The predictive model(s) used by the outcome prediction module can be created in a variety of ways as exemplified herein using independent variables such as demographic variables, physiologic variables, clinical variables, and hospital laboratory test results. The model(s) used by the outcome prediction module can be validated as demonstrated herein.

In an exemplary embodiment, the outcome prediction module can be configured to provide a user of the system with at least one predicted outcome for each of a plurality of different therapeutic agents for a patient, thereby facilitating comparison between the different therapeutic agents. In other words, the outcome prediction module can be configured to facilitate comparison between different possible outcomes of treatment with different therapeutic agents for a patient, thereby helping the user determine which, if any, of the treatments may be valuable to pursue as a treatment option for the patient. Similarly, the outcome prediction module can be configured to provide a user of the system with at least one predicted outcome for a patient in, or being considered for, a clinical trial, thereby facilitating selection of suitable patients in a phase II, III or IV clinical trial.

As mentioned above, the user of the system can be the patient, a medical practitioner treating the patient, or another user, such as a family member of the patient, a medical student, etc. The outcome prediction module can thus provide information to a variety of people for a variety of different purposes. For non-limiting example, the outcome prediction module can allow a patient to self-evaluate different therapeutic agents, which can help educate the patient about real-world consequences of different treatment options. The patient can therefore ask medical practitioners more informed questions and/or can have more realistic expectations of outcomes.

The outcome prediction module can be configured to read information from and/or write information to any one or more of the databases. In an exemplary embodiment, the outcome prediction module can be configured to write information to the patient data database, such as predicted outcome results for patients. In other words, the outcome prediction module can be configured to store predicted outcome results in the system, thereby allowing a user to retrieve saved predicted outcome results for future consultation, e.g., to allow a patient and medical practitioner to discuss the predicted outcome results in person, to facilitate comparison of treatment results with predicted outcome results, to allow a user to modify some input patient data without having to reenter data in all patient data input fields, etc.

The outcome prediction module can be configured to generate predicted outcomes of treatment in a variety of ways. In an exemplary embodiment, the outcome prediction module can be configured to input patient data regarding a patient entered via the patient data input module and/or stored in the patient data database to a model configured to compare the input patient data to arrive at one or more predicted outcomes for each of one or more different therapeutic agents. The model can be based on historical data and include an equation with coefficients based on historical data, with the input patient data being plugged in as input variables in the equation. As will be appreciated by a person skilled in the art, a model can include any one or more of stratified univariate analysis, multivariable regression models, generalized linear models, and other data mining techniques on multiple data sets. Multivariable models can be based on a variety of variable selection techniques such as forward selection, stepwise selection, backward elimination, least angle regression, all-possible subsets, fully saturated, Bayesian, and any another advanced selection algorithm. Variable selection within these techniques can be based on a variety of statistical measures such as F-tests, the chi-square score statistic, residual mean square error (MSE), coefficient of multiple determination (R2), adjusted coefficient of multiple determination (AdjR2), Akaike's information criterion (AIC), Hannan and Quinn information criterion (HQ), Schwarz criterion (BIC), and Mallow's Cp. In another exemplary embodiment, the outcome prediction module can be configured to input patient data regarding a patient entered via the patient data input module and/or stored in the patient data database and input historical data entered via the historical data input module and/or stored in the historical data database to a model configured to compare the input patient data and the input historical data to arrive at one or more predicted outcomes for one or more therapeutic agents. Inputting historical data can take more time and/or use more processing resources than using an equation based on historical data, but inputting historical data may allow for more recently gathered historical data to be considered in the analysis performed by the outcome prediction module.

The web interface configured to allow users to access the outcome prediction module can have a variety of configurations. The web interface providing one or more predicted outcomes can include a graphical display (e.g., image(s) and, optionally, text). The web interface can, however, provide one or more predicted outcomes as a text-only display. In an exemplary embodiment, the predicted outcome(s) can be displayed in one or more graphs, e.g., bar graphs, line graphs, pie graphs, etc., as graphs can typically be easily understood by non-medically trained users as well as by medically trained users.

The web interface providing one or more predicted outcomes can display predicted outcomes for each of a plurality of different therapeutic agents on a single web page, thereby facilitating comparison between the different types agents. The web interface providing one or more predicted outcomes can be the same web interface configured to allow input of patient data thereto, e.g., after a user submits patient data such as by clicking a “submit” button on a web interface, predicted outcomes can be displayed on the web interface. The user can thus be able to view a comprehensive collection of data, including the input patient data and the predicted outcomes. In some embodiments, a subset of the input patient data can be displayed on a same web interface as the predicted outcomes. Alternatively, the web interface providing one or more predicted outcomes can be a separate web interface from the interface configured to allow input of patient data thereto, e.g., after a user submits patient data such as by clicking a “submit” button on a patient data web interface, a predicted outcomes web interface can be displayed. Providing less information on the web interface, e.g., displaying the predicted outcomes without displaying the input patient data, can help make the web interface less cluttered and thus easier for the user to view.

The invention is further illustrated by the following non-limiting examples.

### EXAMPLE 1

### Methods

The database from the second phase III clinical trial of the E5 anti-endotoxin antibody in sepsis (Bone, et al. (1995) supra) was supplied by XOMA LLC (Berkeley, Calif.). Data from the Synergen 0509 clinical trial of interleukin (IL)-1ra in sepsis (Fisher, et al. (1994) JAMA 271:1836-1843) were supplied by Amgen, Inc. (Thousand Oaks, Calif.). Data from the NORASEPT and NORASEPT II clinical trials (Abraham, et al. (1995) JAMA 273:934-941; Abraham, et al. (1998) Lancet 351:929-923) were supplied by the Bayer Corporation (West Haven, Conn.). Data from the COMPASS clinical trial of PAF-AH in sepsis (Opal, et al. (2004) Crit. Care Med. 32:332-341) were supplied by ICOS Corporation (Seattle, Wash.). The clinical trial database of the CORTICUS study (Sprung, et al. (2008) N. Engl. J. Med. 358:111-124) was supplied by Charles Sprung, M.D. Details of each of these clinical trials are summarized in Table 1.

TABLE 1

| | | | | Year |
|---|---|---|---|---|
| Clinical | | | Entry | Study |
| Trial | Sponsor | Study Drug | Criteria | Ended |
| E5 | XOMA | E5 anti- | Sepsis | 1991 |
| | | endotoxin- | syndrome | |
| | | modified | | |
| | | antibody | | |
| NORASEPT | Bayer | TNFMAb | Sepsis | 1993 |
| | | antitumor | syndrome | |
| | | necrosis factor | | |
| | | monoclonal | | |
| | | antibody | | |
| NORASEPT | Bayer | TNFMAb | Septic shock | 1998 |
| II | | | | |
| 0509 | Synergen | IL-1ra | Modified | 1994 |
| | | | Sepsis | |
| | | | syndrome | |
| COMPASS | ICOS | PAF-AH | Modified | 2004 |
| | | | ACCP/SCCM | |
| | | | consensus | |
| | | | definitions | |
| | | | of sepsis | |
| CORTICUS | Multiple | Hydrocortisone | Modified | 2005 |
| | | (50 mg IV every | ACCP/SCCM | |
| | | 6 hours for 5 | Consensus | |
| | | days) | Definitions | |
| | | | of Septic | |
| | | | Shock | |

No patient-identifying information was included. The NORASEPT and NORASEPT II studies were sequential multi-institutional studies of TNFMAb in severe sepsis and septic shock. All investigations were prospective, randomized, double blind, placebo-controlled phase III clinical trials. In the E5 study, the primary end point was a 30-day all-cause mortality (Bone, et al. (1995) supra). The primary end point in the NORASEPT and NORASEPT II, Synergen 0509, COMPASS and CORTICUS studies was 28-day all-cause mortality (Fisher, et al. (1994) supra; Abraham, et al. (1995) supra; Abraham, et al. (1998) supra; Opal, et al. (2004) supra). Details of these studies were thoroughly described in the articles that reported their results (Bone, et al. (1995) supra; Fisher, et al. (1994) supra; Abraham, et al. (1995) supra; Abraham, et al. (1998) supra; Opal, et al. (2004) supra; Dellinger, et al. (2004) Crit. Care Med. 32:858-873).

In NORASEPT, septic mortality was slightly reduced, but not significantly, among patients with shock at baseline who received the 7.5 mg/kg TNFMAb dosage (Abraham, et al. (1995) supra). In NORASEPT II, therefore, the investigators decided to randomize only patients with septic shock at baseline to either placebo or 7.5 mg/kg TNFMAb (Abraham, et al. (1998) supra). Because the enrollment criteria were otherwise identical, the two studies were considered sufficiently similar to use patient data from NORASEPT II to validate the SMART models developed on NORASEPT.

In the CORTICUS, E5, NORASEPT, IL-1ra, and the preinterim analysis cohort of COMPASS, on HIPAA compliant, prerandomization clinical information from patients in each study for whom complete data sets were available, using multivariate, step-wise logistic regression with all ways elimination (simultaneous forward and backward elimination of nonweighted independent variables), SMART survival models were separately developed for the placebo and active drug groups. For the E5 study, SMART models also predicted drug effects on organ failure or death. Statistical significance at p<0.10 identified potential independent variables and was the threshold for testing them in the final equations, with, conversely, p>0.10 being the threshold for excluding a potential independent variable. These separate survival models for each study, generated separately from the placebo and from the active drug baseline, prerandomization databases, made it possible to test two possible probabilities for each individual patient: the probability of survival for that patient receiving the active study drug and placebo. After the modeling process was completed, prerandomization data from every patient in that study were entered into both equations, and lengthy explorations into the relationship between the placebo and active drug models and their interactions with treatment effects were undertaken to analyze optimum cutoffs for each drug. Beginning with the original consensus definition patient population, this process tested study drug treatment effects in progressively smaller subpopulations, incrementally excluding, always at prerandomization baseline, from each study's efficacy analysis patients whom SMART predicted would survive if they were to receive placebo and/or who would expire if they were to receive the active drug. This exploration was performed for each clinical trial on a theoretically infinite number of cutoff points, with efficacy in reducing septic mortality tested for each study drug in cohorts having mortality rates ranging from 0% to 100%. As patients who were excluded from efficacy analysis at each cutoff point were identified before randomization, the resulting placebo and active drug subgroups were, by definition, equal. With this approach, only subjects who were identified by the SMART models for each study as responsive to the treatment arm were included in outcomes statistics, thereby giving each drug a fair chance to prove its efficacy. Survival-treatment effects were evaluated separately among patients enrolled under consensus definitions and among patients predicted by SMART to respond to each sepsis drug. Mortality was analyzed by Kaplan-Meier statistics (SAS Institute (1994) SAS/STAT User's Guide, Version 6, 4^{th} Ed. Cary, N.C.) as were the E5 results for drug treatment effects on end-organ dysfunction. The E5 and Synergen 0509 results were retrospective, because the Synergen 0556 study database (Opal, et al. (1997) Crit. Care Med. 25:115-123) was not released, and the third phase III clinical trial of E5 versus placebo in sepsis had insufficient data to support the SMART models (Angus, et al. (2000) JAMA 283:1723-1730).

As prospective validation of the SMART models for the TNFMAb molecule, and of the efficacy of the drug, baseline information from NORASEPT II subjects was entered into SMART models from NORASEPT. Then, treatment effects of TNFMAb were assessed among consensus NORASEPT II patients, and, separately, in the SMART cohort.

In the COMPASS clinical trial PAF-AH, modeling was conducted on the 600 patients enrolled for the interim analysis. Then, PAF-AH versus placebo treatment effects were tested prospectively by entering data from the 623 subjects in the second COMPASS interim analysis cohort into the SMART models built upon the first interim group's data.

The X² equation (SAS Institute (1994) supra) was used to ensure that the distribution of baseline discrete variables was equal within each study for placebo versus active drug populations.

### EXAMPLE 2

### Results Using SMART Models

Baseline parameters that were screened as possible independent variables for SMART models that were developed from the CORTICUS, E5, TNFMAb, IL-1ra, and PAF-AH clinical trial databases are listed in Table 2. Nearly, all these demographic, physiologic, clinical, and hospital laboratory data points were captured at prerandomization baseline in each study, always within 24 hours or less before administrations of the study drug. Nearly, all the variables listed were measured at prerandomization baseline in every patient, pursuant to FDA safety-monitoring requirements (Dellinger, et al. (2004) supra; Bone, et al. (1995) supra; Fisher, et al. (1994) supra; Abraham, et al. (1995) supra; Abraham, et al. (1998) supra; Opal, et al. (2004) supra).

TABLE 2

Baseline Observations
APACHE II score
Body surface area
Underlying comorbidities
Cardiovascular
Pulmonary disease
Autoimmune
Hematologic
Hepatic
Neurologic
Renal or bladder
Diabetes mellitus
Cancer
Other endocrine
Immunosuppressive therapy
Sex
Alcoholism
Simplified Acute Physiology Score (SAPS)
Source of infection
Urinary tract
Lungs
Intra-abdominal
Wound
Blood
Central nervous system
Indwelling catheter
Other
Causative microorganism
Diagnostic procedures
Estimated sepsis severity
Blood pressure: systolic,
diastolic, mean
Heart rate
Respiratory rate
Glasgow Coma Scale
Age
Days since admission
Blood work
Serum Electrolytes
Hemoglobin
Hematocrit
White blood cell count
Platelets
Arterial blood gas
FiO₂
Estimated sepsis severity
Race
Major surgery/trauma
Elective
Emergency
Cardiac output
Sequential Organ Failure
Assessment (SOFA) score
Baseline organ failure
Renal
Acute respiratory distress
syndrome (ARDS)
Disseminated intravascular
coagulation (DIC)
Hepatobiliary
Central nervous system
Shock
Abnormal physical examination
Neck
Abdomen
Skin
Extremities
Neurologic
HEENT
Respiratory
Cardiovascular

Independent variables that were weighted components of the SMART models built on the NORASEPT sepsis study are displayed in Table 3.

TABLE 3

| | |
|---|---|
| Placebo Model | TNFMab Model |

| | | Odds | | Odds |
|---|---|---|---|---|
| | p | Ratio | p | Ratio |
| NORASEPT | | | | |
| APACHE II Score | <0.001 | 1.089 | <0.001 | 1.116 |
| PTT | 0.02 | 1.016 | — | — |
| RBC | <0.001 | 0.473 | — | — |
| ROC AUC | 0.777 | | 0.737 | |
| NORASEPT II | | | | |
| Prospectively | | | | |
| validated | | | | |
| models | | | | |
| ROC AUC | 0.727 | | 0.703 | |

SMART models that predicted 28-day all-cause mortality risk were generated separately from the placebo and active drug clinical trial databases, using prerandomization data.

TNFMAb versus placebo treatment effects on 28-day all-cause mortality for NORASEPT and NORASEPT II are respectively displayed in Tables 4 and 5.

TABLE 4

| NORASEPT | |
|---|---|
| Consensus | |
| Definition | SMART cohort |
| Cohort (n = 623) | (n = 205) |

| | Placebo | TNFMAb | Placebo | TNFMAb |
|---|---|---|---|---|
| Total | 308 | 315 | 110 | 95 |
| Dead | 103 | 93 | 52 | 33 |
| Alive | 225 | 222 | 58 | 62 |
| Mortality (%) | 33.4 | 29.5 | 47.2 | 34.7 |
| Absolute* | 3.9 | | 12.6 | |
| Relative* | 11.7 | | 26.6 | |
| P* | | 0.20 | | 0.03 |

*Mortality reduction vs placebo (%).
SMART cohort was identified through analysis of interactions between study drug treatment effects and prerandomization placebo and active drug survival models.

TABLE 5

| | NORASEPT II | | | | | |
|---|---|---|---|---|---|---|
| | Consensus | | | | | |
| | Definition | | SMART | | Non-SMART | |
| | Cohort | | cohort | | Cohort | |
| | (n = 1741) | | (n = 744) | | (n = 997) | |
| | Pla- | | Pla- | | Pla- | |
| | cebo | TNFMAb | cebo | TNFMAb | cebo | TNFMAb |
| Total | 863 | 878 | 371 | 373 | 492 | 505 |
| Dead | 379 | 360 | 184 | 158 | 195 | 202 |
| Alive | 484 | 518 | 187 | 215 | 297 | 303 |
| Mortality | 43.9 | 41.0 | 49.6 | 42.4 | 39.6 | 40.0 |
| (%) | | | | | | |
| Absolute* | 2.9 | | 7.2 | | 0 | |
| Relative* | 6.6 | | 14.5 | | 0 | |
| P* | | 0.15 | | 0.02 | | |

*Mortality reduction vs placebo (%).
SMART cohort was identified through analysis of interactions between study drug treatment effects and prerandomization placebo and active drug survival models.

For the 623 patients in NORASEPT, mortality was 33.4% placebo and 29.5% TNFMAb (3.9% absolute reduction; 11.7% relative to placebo; p=0.20). In the SMART cohort, placebo mortality was 47.3% and 34.7% TNFMAb (12.6% absolute; 26.9% relative to placebo; p=0.03). For NORASEPT II, mortality was 43.9% placebo and 41.0% TNFMAb (2.9% absolute; 6.6% relative to placebo; p=0.15). In the NORASEPT II SMART cohort, 28-d mortality was 49.6% placebo and 42.4% TNFMAb (7.2% absolute and 14.5% relative to placebo; p=0.02).

Independent variables in SMART models for E5 antiendotoxin antibody are displayed in Table 6.

TABLE 6

| | Odds Ratio Estimates - |
|---|---|
| Independent Variable | 95% Wald Confidence Limits |
| APACHE II Score | 1.039-1.144 |
| Urinary tract source of infection | 0.222-0.727 |
| Lung source of infection | 0.920-4.889 |
| Respiratory rate | 1.008-1.071 |
| Diastolic blood pressure | 0.951-0.987 |
| DIC | 1.344-16.808 |
| Age | 1.027-1.067 |
| Neurologic comorbidity | 1.341-5.185 |
| Acute CNS dysfunction | 0.140-0.517 |
| ARDS | 3.702-18.304 |
| Hepatobiliary dysfunction | 1.734-19.037 |

CNS, central nervous system.
SMART models that predicted 28-d all-cause mortality risk were generated separately from the placebo and active drug clinical trial databases, using prerandomization data.

Treatment effects on 30-day all-cause mortality for E5 versus placebo are displayed in Table 7.

TABLE 7

| | |
|---|---|
| Consensus | |
| Definition | SMART cohort |
| Cohort (n = 759) | (n = 388) |

| | E5 | Placebo | E5 | Placebo |
|---|---|---|---|---|
| Total | 390 | 369 | 201 | 187 |
| Dead | 102 | 101 | 16 | 32 |
| Alive | 288 | 268 | 185 | 155 |
| Mortality (%) | 26.2 | 27.4 | 8.0 | 17.1 |
| Absolute* | 1.2% | | 9.1% | |
| Relative* | 4.4% | | 53.2% | |
| P* | | 0.0747 | | 0.006 |

*Mortality reduction vs. placebo (%).
SMART cohorts were identified through analysis of interactions between study drug treatment effects and prerandomization placebo and active drug survival models.

Organ failure/death in severe sepsis and septic shock for E5 versus placebo are displayed in Table 8.

TABLE 8

E5 vs. Placebo p values

| | Consensus cohort | SMART cohort |
|---|---|---|
| | (n = 759) | (n = 388) |
| ARDS | 0.43 | 0.01 |
| Hepatobiliary | 0.65 | 0.03 |
| Renal | 0.81 | 0.22 |
| CNS | 0.20 | 0.02 |
| DIC | 0.54 | 0.002 |
| Shock | 0.97 | 0.04 |

SMART cohorts were identified through analysis of interactions between study drug treatment effects and prerandomization placebo and active drug survival models.

In the consensus E5 population, placebo mortality was 27.4% and E5 26.2% (1.2% absolute; 4.4% relative to placebo; p=0.747). In the E5 SMART cohort, placebo mortality was 17.1% and E5 8.0% (9.1% absolute; 53.2% relative to placebo; p<0.01).

Independent variables of SMART models from the Synergen 0509 clinical trial of IL-1ra in sepsis are displayed in Table 9.

TABLE 9

| | Odds Ratio Estimates - 95% |
|---|---|
| Independent Variable | Wald Confidence Limits |
| Placebo model results (n = 302)* | |
| ARDS | 0.169-0.621 |
| DIC | 0.135-0.616 |
| Mean arterial pressure | 1.007-1.047 |
| Temperature | 1.082-1.634 |
| Arterial pH | 1.673-5.427 |
| BUN | 0.967-0.990 |
| FiO₂ | 0.990-0.999 |
| High-dose IL-1ra model results (n = 293)† | |
| Cardiovascular | 0.264-0.934 |
| Age | 0.965-0.998 |
| Systolic blood pressure | 1.003-1.034 |
| Respiratory infection | 0.288-0.895 |
| Urinary tract infection | 1.993-25.933 |
| BUN | 0.978-0.998 |
| Low-dose IL-1ra model results (n = 298)‡ | |
| ARDS | 0.193-0.738 |
| DIC | 0.138-0.595 |
| Acute Renal Failure | 0.215-0.708 |
| Vasco | 0.274-0.872 |
| Age | 0.956-0.989 |
| HEENT abnormal | 0.214-0.717 |
| Abdomen abnormal | 0.328-1.124 |
| Neurological abnormal | 0.361-1.119 |
| Extremities/joint abnormal | 0.320-1.009 |

*ROC AUC = 0.822.
†ROC AUC = 0.762.
‡ROC AUC = 0.776.
SMART models that predicted 28-d all-cause mortality risk were generated separately from the placebo and active drug clinical trial databases, using prerandomization data.

Treatment effects of IL-1ra versus placebo on 28-day all-cause mortality are displayed in Tables 10-12.

TABLE 10

| | Consensus Definition Cohort | | |
|---|---|---|---|
| | Placebo | Low Dose | High Dose |
| Total | 298 | 290 | 289 |
| Dead | 101 | 93 | 86 |
| Alive | 197 | 197 | 203 |
| Mortality (%) | 33.9 | 32.1 | 29.8 |
| Absolute* | | 1.8 | 4.1 |
| Relative* | | 4.3 | 12.1 |
| P* | | 0.618 | 0.282 |

*Mortality change vs. placebo (%).
SMART cohorts were identified through analysis of interactions between study drug treatment effects and prerandomization placebo and active drug survival models.

TABLE 11

| | SMART Cohort High Dose | | | | | |
|---|---|---|---|---|---|---|
| | Pla- | High | Pla- | High | Pla- | High |
| | cebo | Dose | cebo | Dose | cebo | Dose |
| Total | 176 | 181 | 133 | 123 | 77 | 72 |
| Dead | 85 | 66 | 74 | 43 | 52 | 29 |
| Alive | 91 | 115 | 59 | 80 | 25 | 43 |
| Mortality | 48.3 | 36.5 | 55.6 | 35.0 | 67.5 | 40.3 |
| (%) | | | | | | |
| Absolute* | | 11.8 | | 20.6 | | 27.2 |
| Relative* | | 24.4 | | 37.1 | | 40.3 |
| P* | | 0.024 | | 0.0009 | | 0.0008 |

*Mortality change vs. placebo (%).
SMART cohorts were identified through analysis of interactions between study drug treatment effects and prerandomization placebo and active drug survival models.

TABLE 12

SMART Cohort Low Dose

| | Placebo | Low dose | Placebo | Low dose |
|---|---|---|---|---|
| Total | 169 | 165 | 61 | 54 |
| Dead | 79 | 56 | 38 | 14 |
| Alive | 90 | 109 | 23 | 40 |
| Mortality (%) | 46.7 | 35.9 | 62.3 | 25.9 |
| Absolute* | | 10.8 | | 36.4 |
| Relative* | | 23.1 | | 58.4 |
| P* | | 0.017 | | <0.0001 |

*Mortality change vs. placebo (%).
SMART cohorts were identified through analysis of interactions between study drug treatment effects and prerandomization placebo and active drug survival models.

In sepsis syndrome patients (n=877), mortality was 33.9% placebo, 32.1% for 1.0 mg/kg/h IL-1ra (1.8% absolute; 5.3% relative; p=0.6178), and 29.8% for IL-1ra 2.0 mg/kg/h (4.1% absolute; 12.1% relative; p=0.2824). In one SMART cohort (59.2%/62.6% of placebo/IL-1ra consensus populations), placebo mortality was 48.3%, versus IL-1ra, at 2.0 mg/kg/h, 36.5% (11.8% absolute; 24.4% relative; p=0.024). In a more IL-1ra-specific SMART cohort (44.6%/42.6% of placebo/IL-1ra consensus populations), placebo mortality was 55.6% versus 35.0% IL-1ra (20.6% absolute; 37.1% relative; p<0.001). In a third SMART cohort (25.8%/24.9% of placebo/IL-1ra consensus populations), placebo mortality was 67.5% versus 40.3% IL-1ra (27.2% absolute; 37.1% relative; p<0.001).

For IL-1ra 1.0 mg/kg/h, in a SMART cohort (56.7%/56.9% of placebo/IL-1ra consensus populations), placebo mortality was 46.7% versus 35.0% IL-1ra (10.8% absolute; 23.1% relative; p=0.017). Another SMART cohort (20.5%/18.6% of placebo/IL-1ra consensus populations) had placebo mortality 62.3% versus 25.9% IL-1ra (36.4% absolute; 58.4% relative; p<0.0001).

Independent variables for SMART models from the ICOS COMPASS clinical trial are listed in Table 13.

TABLE 13

| | Odds Ratio Estimates - 95% |
|---|---|
| Independent Variable | Wald Confidence Limits |
| Placebo* | |
| Mechanical ventilator | 0.066-0.412 |
| APACHE II score | 1.049-1.171 |
| Multiple organ dysfunction score | 1.006-1.306 |
| Eosinophil count | 0.004-0.062 |
| PAF-AH† | |
| Mechanical ventilator | 0.066-0.412 |
| Multiple organ dysfunction score | 1.006-1.171 |

*ROC AUC = 0.708.
†ROC AUC = 0.788.
SMART models that predicted 28-d all-cause mortality risk were generated separately from the placebo and active drug clinical trial databases, using prerandomization data.

PAF-AH versus placebo treatment effects on 28-day all-cause mortality are displayed in Tables 14 and 15.

TABLE 14

| | COMPASS I Clinical Trial | | | |
|---|---|---|---|---|
| | Consensus Definition | | SMART Cohort I | |
| | Cohort (n = 587) | | (n = 251) | |
| | Placebo | PAF-AH | Placebo | PAF-AH |
| Total | 304 | 283 | 130 | 121 |
| Dead | 68 | 65 | 23 | 35 |
| Alive | 236 | 218 | 107 | 86 |
| Mortality (%) | 22.4 | 22.9 | 17.7 | 28.9 |
| Absolute* | 0.5 | | 11.2 | |
| Relative* | 2.2 | | 63.3 | |
| P* | 0.921 | | 0.039 | |

*Mortality change vs. placebo (%).
SMART cohorts were identified through analysis of interactions between study drug treatment effects and prerandomization placebo and active drug survival models.

TABLE 15

| | COMPASS II Clinical Trial | | | |
|---|---|---|---|---|
| | Consensus Definition | | SMART Cohort II | |
| | Cohort (n = 540) | | (n = 244) | |
| | Placebo | PAF-AH | Placebo | PAF-AH |
| Total | 255 | 285 | 119 | 125 |
| Dead | 66 | 73 | 38 | 27 |
| Alive | 189 | 212 | 81 | 98 |
| Mortality (%) | 25.9 | 25.6 | 31.9 | 21.6 |
| Absolute* | 0.3 | | 10.3 | |
| Relative* | 1.1 | | 32.3 | |
| P* | 1.000 | | 0.0551 | |

*Mortality change vs. placebo (%).
SMART cohorts were identified through analysis of interactions between study drug treatment effects and prerandomization placebo and active drug survival models.

In the consensus COMPASS population (COMPASS I), placebo mortality was 22.4% versus 22.9% for PAF-AH (0.5% absolute survival increase; 2.2% relative; p=0.924). The SMART cohort of COMPASS I had placebo mortality 17.7% versus PAF-AH 28.9% (11.2% absolute increase in septic mortality versus placebo; 63.3% relative; p=0.039). The COMPASS I SMART models and PAF-AH treatment effects were tested prospectively on the COMPASS II population that followed COMPASS I up to the second and final interim analysis. In the COMPASS II consensus population (n=540), placebo mortality was 25.9% versus 25.6% for PAF-AH. In the SMART COMPASS II cohort (n=244), placebo mortality was 31.9% versus 21.6% for PAF-AH (10.3% absolute reduction in mortality; 32.3% relative; p=0.0551).

Independent variables that were weighted components of the SMART models built on the CORTICUS sepsis study are listed in Tables 16 (placebo model) and 17 (treatment model). There were 500 patients in the CORTICUS database. Due to missing values and values in error, there were only 446 (89%) patients that were analyzable.

TABLE 16

| | | | | | Odds Ratio |
|---|---|---|---|---|---|
| | | | | | Estimates - |
| | Estimate | Wald | | | 95% Wald |
| | (Std. | Chi- | p- | Point | Confidence |
| Parameter | Error) | Square | value | Estimate | Limits |
| Intercept | −4.0449 | 29.6260 | <0.0001 | | |
| | (0.7431) | | | | |
| Hypertension | 0.6384 | 4.2401 | 0.0395 | 1.893 | 1.031-3.476 |
| | (0.3100) | | | | |
| Respiratory | 0.0441 | 6.8249 | 0.0090 | 1.045 | 1.011-1.080 |
| Rate | (0.0169) | | | | |
| SAPS | 0.0411 | 15.1477 | <0.0001 | 1.042 | 1.042-1.021 |
| | (0.0105) | | | | |

N = 224;
AUC = 0.714.

TABLE 17

| | | | | | Odds Ratio |
|---|---|---|---|---|---|
| | | | | | Estimates - |
| | Estimate | Wald | | | 95% Wald |
| | (Std. | Chi- | p- | Point | Confidence |
| Parameter | Error) | Square | value | Estimate | Limits |
| Intercept | −6.9042 | 29.4342 | <0.0001 | | |
| | (1.2726) | | | | |
| Age | 0.0421 | 10.7968 | 0.0010 | 1.043 | 1.017-1.069 |
| | (0.0128) | | | | |
| PaCO2 High | 0.0460 | 10.0603 | 0.0015 | 1.047 | 1.018-1.077 |
| mmHg | (0.0145) | | | | |
| BE low | −0.0741 | 7.4116 | 0.0065 | 0.929 | 0.880-0.979 |
| mmol l | (0.0272) | | | | |
| 24 before | 0.0237 | 6.1457 | 0.0132 | 1.024 | 1.005-1.043 |
| Total SAPS | (0.00956) | | | | |

N = 222;
AUC = 0.734.

Hydrocortisone versus placebo treatment effects for CORTICUS patients are displayed in Table 18.

TABLE 18

| | Consensus | | | | | |
|---|---|---|---|---|---|---|
| | Definition | | SMART | | Non-SMART | |
| | Cohort | | Cohort | | Cohort | |
| | (n = 446) | | (n = 421) | | (n = 25) | |
| | Placebo | HC | Placebo | HC | Placebo | HC |
| Total | 224 | 222 | 212 | 209 | 12 | 13 |
| Alive | 153 | 144 | 148 | 141 | 5 | 3 |
| Dead | 71 | 78 | 64 | 68 | 7 | 10 |
| Mortality | 31.70 | 35.14 | 30.19 | 32.54 | 58.33 | 76.92 |
| (%) | | | | | | |
| Absolute* | | −3.44 | | −2.35 | | |
| Relative* | | −10.85 | | −7.78 | | |
| P* | | 0.6019 | | 0.1696 | | 0.2636 |

*Mortality change vs. placebo (%).
HC, hydrocortisone.
SMART cohorts were identified through analysis of interactions between study drug treatment effects and prerandomization placebo and active drug survival models.

Independent variables that were weighted components of the SMART models built on the corticotrophin non-responder database of CORTICUS are listed in Tables 19 (placebo model) and 20 (treatment model).

TABLE 19

| | | | | | Odds Ratio |
|---|---|---|---|---|---|
| | | | | | Estimates - |
| | Estimate | Wald | | | 95% Wald |
| | (Std. | Chi- | p- | Point | Confidence |
| Parameter | Error) | Square | value | Estimate | Limits |
| Intercept | −3.3199 | 17.8538 | <0.0001 | | |
| | (0.7857) | | | | |
| CRF | 1.2364 | 3.0882 | 0.0789 | 3.443 | 0.867-13.671 |
| | (0.7035) | | | | |
| SAPS | 0.0439 | 10.4859 | 0.0012 | 1.045 | 1.017-1.073 |
| | (0.0136) | | | | |

N = 107;
AUC = 0.728.

TABLE 20

| | | | | | Odds Ratio |
|---|---|---|---|---|---|
| | | | | | Estimates - |
| | Estimate | Wald | | | 95% Wald |
| | (Std. | Chi- | p- | Point | Confidence |
| Parameter | Error) | Square | value | Estimate | Limits |
| Intercept | 75.8534 | 9.0511 | 0.0026 | | |
| | (25.2130) | | | | |
| SAPS | 0.0587 | 11.6183 | 0.0007 | 1.060 | 1.025-1.097 |
| | (0.0172) | | | | |
| Temperature | −0.8936 | 10.9555 | 0.0009 | 0.409 | 0.241-0.695 |
| | (0.2700) | | | | |
| Norepi- | 2.4155 | 8.4438 | 0.0037 | 11.196 | 2.195-57.101 |
| nephrine | (0.8313) | | | | |
| pH low | −6.4081 | 5.1013 | 0.0239 | 0.002 | <0.001-0.429 |
| | (2.8372) | | | | |
| Hypertension | 1.0664 | 3.9387 | 0.0472 | 2.905 | 1.013-8.327 |
| | (0.5373) | | | | |

N = 108;
AUC = 0.866.

Hydrocortisone versus placebo treatment effects of septic shock survival among CORTICUS patients who did not respond to corticotrophin are tabulated in Table 21.

TABLE 21

| | Consensus | | | | | |
|---|---|---|---|---|---|---|
| | Definition | | SMART | | Non-SMART | |
| | Cohort | | Cohort | | Cohort | |
| | (n = 216) | | (n = 168) | | (n = 38) | |
| | Placebo | HC | Placebo | HC | Placebo | HC |
| Total | 96 | 119 | 82 | 86 | 14 | 24 |
| Dead | 66 | 66 | 57 | 63 | 9 | 3 |
| Alive | 30 | 44 | 25 | 23 | 5 | 21 |
| Mortality | 31.25 | 40.00 | 30.49 | 26.74 | 35.71 | 87.50 |
| (%) | | | | | | |
| Absolute* | | −8.75 | | 3.75 | | |
| Relative* | | −28 | | 12.3 | | |
| P* | | 0.3209 | | 0.2973 | | |

*Mortality change vs. placebo (%).
HC, hydrocortisone.
SMART cohorts were identified through analysis of interactions between study drug treatment effects and pre-randomization placebo and active drug survival models

When all CORTICUS patients were included in analyses, among consensus septic shock patients, hydrocortisone mortality was 35.14%, compared with placebo mortality 31.7% (p=0.6019). In the overall SMART cohort from CORTICUS, hydrocortisone and placebo moralities were 43.54 and 30.19, respectively (p=0.1696). Among corticotropin non-responders, overall hydrocortisone/ placebo mortality was 40.0%/31.25%, respectively, an −8.75% adverse hydrocortisone treatment effect (p=0.3209). In the SMART coticotropin non-responder group, hydrocortisone/placebo mortality was 26.74% versus 30.49%, respectively (p=0.2973).

There were few weighed independent variables that were common between the five clinical trials in the SMART placebo models. Placebo models from the IL-1ra and E5 studies had disseminated intravascular coagulation (DIC) and acute respiratory distress syndrome (ARDS) as significantly weighted independent variables. APACHE (Acute Physiology and Chronic Health Evaluation) II score was common to the NORASEPT and COMPASS clinical trials. No other independent variables factored significantly in more than one SMART placebo model.

### EXAMPLE 3

### Application of SMART Models

In the XOMA E5 sepsis clinical trial, SMART discovered patients among whom E5 not only improved survival but also reduced organ failure. Subjects enrolled by consensus definitions alone received only a nonsignificant 1.4% absolute survival benefit from E5. In the SMART cohort, however, which included 51% of the consensus population, E5 reduced mortality by 9.1% absolute, 53.2% relative to placebo. In the SMART cohort, placebo mortality was only 17.1%, more than 10% lower than in the parent consensus definition population. Logically, one might expect gram-negative infection to have been a weighted independent variable in SMART models for an anti-endotoxin antibody, but infecting bacteriology did contribute to these equations. On the surface, these findings also seem inconsistent with the results of the MEDIC study (Marshall, et al. (2004) J. Infect. Dis. 190:527-534), which reported strong correlations between increased circulating endotoxin levels and high APACHE II, MOD, and SOFA scores, shock, decreasing partial pressure of oxygen in arterial blood/fractional inspired oxygen ratio, and leucopenia or leukocytosis. The results of this study, specifically the finding of E5-responsive patients in a lower mortality subgroup, presumably, therefore, with low-circulating endotoxin (Marshall, et al. (2004) supra), suggest that endotoxin levels alone might not predict treatment effects for anti-endotoxin strategies. It may be that E5 succeeded here by SMART's incorporating the septic pathophysiology of individual patients into the subject selection data mix.

Another interesting observation was that E5 reduced septic mortality only in lower acuity patients, with placebo mortality only 17.1%. This contrasts strikingly with the results of the Phase 2 trial of eritoran tetrasodium (E5564), a toll-like receptor 4 antagonist that interferes with endotoxin signaling (Tidswell, et al. (2010) Crit. Care Med. 38:72-83). In that investigation, a nonsignificant trend toward lower septic mortality was seen in high-dose eritoran subjects with high APACHE II predicted risk of mortality. These results indicate that for each truly effective molecule in sepsis therapy, there are patients whose host-inflammatory responses to infection are matched biologically to that drug, and who, therefore, are specifically able to benefit from it. Apparently, even different anti-endotoxin interventions have different target populations. It follows, logically, then, that the true target populations for different sepsis therapies should vary significantly, according to the mechanism of action of each molecule. The low mortality therapeutic niche identified here for E5 could be confirmed prospectively. Unfortunately, the third E5 sepsis investigation did not capture data sufficient to support the E5 SMART models, and the SMART uncovered also a significant E5 treatment effect on organ failure. Although E5 had no significant effects on organ failure or shock in the consensus population, among SMART E5 responders, ARDS, hepatobiliary failure, cerebral dysfunction, DIC, and shock were reduced dramatically findings here, therefore, could not be validated prospectively (Opal, et al. (1997) Crit. Care Med. 25:115-123).

A clinically significant discovery of this investigation was the unprecedented, extremely high reduction of septic mortality among SMART patients by IL-1ra. Compared with the sepsis syndrome population, in which high-dose IL-1ra reduced mortality by only 4.1% versus placebo, among patients identified by SMART as able to benefit from the study drug, IL-1ra improved survival by from 9% up to 50% absolute, in increasingly IL-1ra-specific cohorts. Such dramatically increased septic survival has not been reported for any other drug ever tested in humans. Unfortunately, the SYNERGEN 0556 sepsis clinical trial of IL-1ra (Angus, et al. (2000) JAMA 283:1723-1730), which followed the 0509 study and was nearly identical to it, was not made available to validate prospectively the SMART/IL-1ra models and the IL-1ra efficacy in sepsis seen here. Considering the life-saving potential of IL-1ra seen here, clinical development of this drug for sepsis should be revisited.

Results of SMART retrospective, post hoc analyses in sepsis, and the efficacy of successful drugs, should be validated prospectively in populations of like patients who were not included in the equation-building process. This was accomplished for SMART models based on NORASEPT. In the post hoc phase, survival benefits of TNFMAb in NORASEPT were improved from 3.9% in consensus patients, to 12.6% in the SMART-identified cohort. Then, baseline raw data from NORASEPT II patients was entered into the SMART equations from NORASEPT. In the SMART cohort of NORASEPT II, TNFMAb lowered septic shock mortality significantly, as it had done in NORASEPT SMART group. These results validated prospectively the predictive power of SMART models from NORASEPT and established TNFMAb efficacy in reducing septic mortality.

SMART prognostic models from the population of the first interim analysis of the COMPASS study of PAF-AH in sepsis also were validated prospectively, using the second and final interim analysis cohort of that clinical trial. Septic mortality was increased significantly compared with placebo among active PAF-AH subjects in the SMART modeling cohort of COMPASS. When data from subjects of the second COMPASS interim analysis group were entered into the SMART models, increased PAF-AH mortality was not confirmed, but, conversely, neither was a significant beneficial effect identified. One might speculate that application of the SMART approach to the first interim analysis data of COMPASS would have resulted in termination of that investigation earlier, with significant savings of research dollars, and, possibly, of adverse drug effects among study subjects.

The addition of SMART statistical analysis to the CORTICUS clinical trial database did not identify a sub-population of CORTICUS patients among whom hydrocortisone reduced septic shock mortality. Rather, in the overall CORTICUS population, the 3.4% increased mortality of the hydrocortisone arm, versus placebo, was reduced immeasurably to a negative 2.5% among the best hydrocortisone-responsive group SMART could find. Among the CORTICUS target cohort of patients in septic shock who did not respond to corticotropin adrenal stimulation, and who, theoretically, would be responsive to stress doses of exogenous steroids, in consensus definition septic shock patients, the mortality rate was 8.75% higher in the hydrocortisone arm than in the placebo group. While SMART models generated from the CORTICUS corticotrophin non-responders identified patients among whom hydrocortisone did improve septic shock survival by 3.75%, this was not a statistically significant treatment advantage. In the face of SMART uncovering groups of individual septic patients within the E5, TNFMAb and IL-1-ra clinical trials wherein each of these drugs reduced septic mortality significantly, one must conclude that hydrocortisone, even when matched to septic shock patients who may be most responsive to it, has no salubrious effect on the death rate in sepsis. Considering in addition the previous reports of increased adverse effects of these drugs in sepsis, including super-infections and augmented mortality with renal failure (Bone, et al. (1987) New Engl. J. Med. 317:653-59; The veterans Administration Systemic Sepsis Cooperative Study Group (1987) New Engl. J. Med. 317:659-655), the results of this study indicate that corticosteroids are not effective adjuvant regimens in septic shock.

From this study, SMART can be used to facilitate clinical development of new therapeutic molecules for sepsis, such as other anti-TNF strategies (Pulmonary Reviews.com (2000) supra; Rice, et al. (2006) Crit. Care Med. 34:2271-2281) anti-endotoxin interventions (Bone, et al. (1995) Crit. Care Med. 23:994-1006; Tidswell, et al. (2010) Crit. Care Med. 38:72-83; Greenman, et al. (1991) JAMA 266:1097-1102) PAF interventions (Dhainaut, et al. (1995) Abst. Am. J. Respir. Crit. Care Med. 151:A447; Dhainaut, et al. (1997) Crit. Care Med. 25:115-123) or restoring coagulation homeostasis (Bernard, et al. (2001) supra), among others. SMART equations derived from Phase II databases could facilitate protocol development for Phase III clinical trials of novel therapies. Similarly, SMART evaluation of completed Phase III investigations could assist in confirmatory study design. Ultimately, SMART interactions with novel drugs may be able to guide bedside management of septic patients, supplemental to clinical judgment and consensus sepsis definitions screening.

Considering the multiple clinical trials testing IL-ra, anti-endotoxin, and anti-TNF regimens that have failed to reduce septic mortality (Pulmonary Reviews.com (2000) supra; Bone, et al. (1995) supra; Fisher, et al. (1994) JAMA 271:1836-1843; Abraham, et al. (1995) JAMA 273:934-941; Abraham, et al. (1998) Lancet 351:929-932; Opal, et al. (2004) supra; Rice, et al. (2006) supra), the results of this investigation indicate that enrollment criteria for such studies should be reconsidered. Certainly, the concept of designing a confirmatory clinical trial on the basis of subgroup analysis from a previous study has been discredited. This is evidenced in the failure of NORASEPT II (Abraham, et al. (1998) supra), wherein shock was added at entry, based on a nonsignificant trend toward anti-TNF efficacy observed in the preceding NORASEPT investigation (Abraham, et al. (1995) supra). In the sequential clinical trials of the E5 antibody, a trend toward efficacy among patients without shock in the first study led to excluding shock in the second study (Bone, et al. (1995) supra; Greenman, et al. (1991) supra). The second IL-1ra sepsis clinical trial (Angus, et al. (2000) JAMA 283:1723-1730) added organ failure and increased APACHE III risk of death as entry criteria, because post hoc analysis suggested a correlation between them and drug treatment responses. All three studies failed to reduce septic mortality. Similarly, severity of illness scores, including APACHE II scoring (Bernard, et al. (2001) supra; Tidswell, et al. (2010) Crit. Care Med. 38:72-83), and/or the presence of DIC (Abraham, et al. (1995) supra) or ARDS (Pittet, et al. (1999) Am. J. Respir. Crit. Care med. 160:852-857), while attractive as single, commonly understood screening measurements, also have not panned out as patient identification tools for predicting anti-TNF and anti-endotoxin treatment responses. Even though APACHE II was an independent variable in SMART survival models for both the E5 and TNFMAb populations, and DIC, and ARDS figured in the E5 SMART modeling, they contributed only to building the tools that identified individual septic pathophysiology. None of these factors directly predicted treatment response. Therefore, as the CytoFab anti-TNF molecule (Rice, et al. (2006) supra) and eritoran tetrasodium (Tidswell, et al. (2010) supra) move from Phase II studies to Phase III confirmatory clinical trials, SMART finds application in supplementing patient identification if standard clinical definitions of sepsis, severity of illness, shock, DIC, or ARDS are to be entry criteria.

SMART may identify also patients for whom sepsis study drugs are ineffective, or even detrimental. During the current study, this was manifested in the preinterim analysis cohort of the COMPASS clinical trial (Opal, et al. (2004) supra), wherein PAF-AH increased septic mortality significantly among a SMART-predicted group. One might speculate that if SMART had been applied to the Phase II PAF-AH database, or even at the first Phase III interim analysis, then COMPASS could have been ended earlier, saving hundreds of subjects from the risk of possible adverse clinical effects.

The results of this study reiterate that the traditional definitions of severe sepsis and septic shock (Bone, et al. (1987) supra; Bone, et al. (1989) supra; Bone, et al. (1992) supra), when used as entry criteria for clinical trials, do not match responsive patients with study drugs that are biologically appropriate to their host pathophysiologies. Therefore, under consensus definition enrollment, new therapies for sepsis are denied a fair chance to prove their efficacy. So many patients are enrolled who would recover on placebo, and who would expire even on active drug, that the true treatment effects of even the most potent sepsis drugs are diluted. Good drugs fail because they are studied in the wrong patients. Then, they are abandoned by the pharmaceutical industry and never reach biologically appropriate patients whom they might save. After nearly three decades of clinical trials that failed because patients were entered through consensus definitions of sepsis, SMART now provides an alternative approach to selecting subjects for these studies.

SMART is an analytic approach that uses conventional statistical techniques and is applicable universally across the gamut of sepsis clinical trials. SMART can be used alone or as a supplement to consensus sepsis definitions given the prevalence of consensus criteria in sepsis clinical trials. Because each novel intervention for sepsis has its own unique mechanism of action, it follows that the host biology of treatment-responsive patients also is unique for each molecule. Therefore, weighted independent variables in the SMART models for E5, for example, are not the same as those for TNFMAb, IL-1ra, or PAF-AH. In addition, clinical factors that would seem to have obvious relevance to sepsis or to a specific drug, such as age, illness acuity, shock, or microbiology, might not pan out as significant independent variables in SMART modeling. Rather, by avoiding preconceived notions of which parameters might predict treatment success, SMART allows the host-inflammatory response to infection of each patient to interact with study drug mechanism of action, thereby building predictive models that match patients to drugs, accurately and objectively. Thus, by the very nature of the SMART approach, SMART is a dynamic process that ferrets out the important temporal interactions within each clinical trial database. The results of this study indicate that the SMART approach works across a variety of therapeutic agents in sepsis clinical trials.

Interestingly, the independent variables for the placebo survival models also varied considerably among the clinical trials analyzed in this study. One might expect, logically, that, at least the placebo patients from different sepsis investigations would be similar, statistically. However, one must realize that sepsis clinical trial entry criteria, while similar in concept, were not uniform in specifics among the studies analyzed here. Thus, NORASEPT, E5, IL-1ra, and COMPASS placebo survival models required varying independent variables, secondary to actual clinical differences in the study populations.

The results here demonstrate SMART's ability to identify objectively patients who can benefit from novel interventions in severe sepsis and septic shock, using readily available prerandomization clinical information, Given the results herein, SMART is also of use in developing predictive models for patients who can respond to molecules that currently are in active clinical development. Whether those models are built on Phase II databases, or as retrospective analyses of completed Phase III clinical trials, when they are used in subsequent confirmatory investigations, it is expected that SMART will give good drugs a fair chance to demonstrate efficacy in sepsis. Moreover, when treatments come into clinical use, SMART finds use in guiding physicians at the bedside, supplemental to consensus sepsis definition screening and to clinical judgment, toward optimizing their efficacy among septic patients in real time.

What is claimed is:

1. A method for treating a systemic inflammatory condition, comprising:
(a) determining whether a patient with a systemic inflammatory condition will respond positively to a therapeutic agent that inhibits tumor necrosis factor, inhibits endotoxin activity, inhibits interleukin-1 receptor, or degrades platelet-activating factor and oxidized phospholipids by
(i) receiving data regarding the patient, the data including one or more baseline parameters of the patient, wherein said baseline parameters comprise demographic variables, physiologic variables, gene expression profiles and results of hospital laboratory tests, wherein
said demographic variables are selected from the group consisting of age, sex, race, comorbidities, and medications;
said physiologic variables are selected from the group consisting of height, weight, temperature, MAP, heart rate, diastolic blood pressure, systolic blood pressure, mechanical ventilation, respiratory rate, pressure support, positive end-expiratory pressure, systemic vascular resistance, cardiac index, pulmonary capillary wedge pressure, blood count, prothrombin time, partial thromboplastin time, fibrin degradation products and D-dimer, urine output, chest X-rays, and bacterial cultures;
said gene expression profiles consist of upregulation or down regulation of expression of one or more protein kinases, growth factors, hormones, enzymes, chemokines, cytokines, receptors, transcription factors, zinc fingers, structural proteins, inflammatory mediators, cell cycle regulators, immune function genes, antimicrobial genes, extracellular matrix and remodeling genes, carbohydrate metabolism genes, and fatty acid metabolism genes; and
said results of hospital laboratory tests are selected from the group consisting of levels of albumin, alkaline phosphatase, ALT, AST, BUN, calcium, cholesterol, creatinine, GGT, glucose, hematocrit, hemoglobin, MCH, MCV, MCHC, phosphorus, platelet count, potassium, total protein, PT, PTT, RBC, sodium, total bilirubin, triglycerides, uric acid, WBCL, base deficit, pH, PaO₂, SaO₂, FiO₂, chloride, and lactic acid;
(ii) generating from the baseline parameters a systemic mediator-associated response test (SMART) profile for the patient;
(iii) using multivariate regression analysis and stepwise regression analysis, to predict an outcome for treatment of the patient's systemic inflammatory condition with the therapeutic agent based at least on the SMART profile and on historical data regarding outcomes of said therapeutic agent administered to a plurality of patients in a phase II or phase III clinical trial; and
(b) administering the therapeutic agent to the patient if the patient is predicted to respond positively to said therapeutic agent.

2. The method of claim 1, wherein the baseline parameters are prerandomization baseline parameters.
